(19) United States
(12) Patent Application Publication
(10) Pub. No.: US 2025/0171595 A1
(43) Pub. Date: May 29, 2025

(54) BIFUNCTIONAL MODIFIED BIOPOLYMER BASED POLYMERS AND HYDROGELS OBTAINABLE FROM SUCH BIFUNCTIONAL MODIFIED BIOPOLYMER BASED POLYMERS

(71) Applicants: Universiteit Gent, Gent (BE); Vrije Universiteit Brussel, Brussel (BE)

(72) Inventors: Jasper Van Hoorick, Roosdaal (BE); Sandra Van Vlierberghe, Sint-Niklaas (BE)

(73) Assignees: Universiteit Gent, Gent (BE); Vrije Universiteit Brussel, Brussel (BE)

(21) Appl. No.: 19/036,745

(22) Filed: Jan. 24, 2025

Related U.S. Application Data
(63) Continuation of application No. 17/058,368, filed on Nov. 24, 2020.

(30) Foreign Application Priority Data
May 31, 2018 (EP) 18175368.2

(51) Int. Cl. C08H 1/06; A61L 26/00; C07K 14/78; C08J 3/075

(52) U.S. Cl. CPC C08H 1/06; A61L 26/0033; A61L 26/0038; A61L 26/008; C07K 14/78; C08J 3/075; C08J 2389/06

(57) ABSTRACT

Bifunctional modified biopolymer based polymer comprise at least one polymer chain comprising n first functional groups and m second functional groups. The first functional groups comprise groups able of being radically cross-linked following a free radical chain-growth polymerisation. The second functional groups comprise groups able to thiol-ene crosslinking. Preferred bifunctional modified biopolymer based polymers comprise bifunctional modified gelatin and bifunctional modified collagen. The invention further relates to a method to prepare such a bifunctional modified biopolymer based polymer and to a method to prepare a hydrogel starting from such bifunctional modified biopolymer based polymer. Furthermore the invention relates to hydrogels obtainable starting from such bifunctional modified biopolymer based polymers and to the use of such hydrogels.

### CROSS-REFERENCES TO RELATED APPLICATIONS

This application is a continuation of U.S. application Ser. No. 17/058,368, filed Nov. 24, 2020, which is a national-stage application under 35 U.S.C. § 371 of International Application No. PCT/EP2019/063995, filed May 29, 2019, which claims benefit of priority to European Patent Application No. 18175368.2, filed May 31, 2018.

### FIELD OF THE INVENTION

The present invention relates to bifunctional modified biopolymer based polymers, in particular bifunctional modified biopolymers such as bifunctional modified gelatin and bifunctional modified collagen, and to a method to prepare such bifunctional modified biopolymer based polymers. The invention further relates to hydrogels obtainable starting from such bifunctional modified biopolymer based polymers and to a method of preparing such hydrogels. Furthermore the invention relates to the use of such hydrogels in biomedical applications as for example in tissue engineering.

### BACKGROUND ART

Gelatin is a nature-derived biopolymer material with excellent cell-interactive properties and the potential to form a hydrogel. It has widespread applications in the food and pharmaceutical industry based on its wide availability and cost-efficiency. As a result, the material has become one of the benchmarks in the field of tissue engineering and biofabrication. However, since gelatin is characterized by an upper critical solution temperature below the physiological temperature (±30° C.), gelatin-based hydrogels are unsuitable for biomedical applications such as tissue engineering. To be suitable in biomedical applications, it is necessary to increase the stability and mechanical properties of gelatin under physiological conditions. Therefore, multiple strategies have emerged to covalently crosslink gelatin. The use of photo-crosslinking strategies is of specific interest as these methods are generally characterized by relatively mild conditions allowing cell encapsulation in the hydrogel. Additionally, certain (high resolution) additive manufacturing techniques, including stereolithography and two photon polymerization (2PP) require photo-crosslinking to structure the material.

The known photo-crosslinking strategies can generally be distinguished into two main categories depending on the crosslinking mechanism: chain-growth polymerization and step-growth polymerization. Historically, the main part of photo-induced gelatin crosslinking strategies are performed using chain-growth polymerization (radical mediated chain-growth photopolymerization). An often reported gelatin derivative in this respect is gelatin-methacrylamide (Gel-MOD or Gel-MA) in which the primary amine groups of gelatin have been functionalized using methacrylic anhydride yielding crosslinkable methacrylamides.

In the last decade, step-growth thiol-ene hydrogels, such as thiol-ene (photo-)click hydrogels have gained increasing interest. They are typically characterized by a higher reactivity and the formation of more homogeneous networks due to their orthogonal nature. Consequently, they exhibit superior compatibility towards cell encapsulation since the reaction is characterized by lower radical concentrations and in contrast to chain-growth hydrogels, the reaction can efficiently take place in the presence of oxygen. To perform thiol-ene chemistry, norbornene functionalities are of particular interest. On the one hand they are not susceptible to competitive homo-polymerization. On the other hand relieving of the ring-strain during reaction with a thiol, in combination with fast subsequent proton transfer, further increases its thiol-ene reactivity.

Gelatin methacrylamide gels (gel-MOD or gel-MA) are generally stiffer compared to thiol-ene hydrogels (gel-NB) due to the nature of the crosslinking. Thiol-ene hydrogels such as gelatin norbornene hydrogels (gel-NB) have the advantage to allow control of the amount of crosslinked functionalities and exhibit improved processing capabilities towards light based additive manufacturing techniques.

Additionally, in general thiol-ene hydrogels (gel-NB) are characterized by a decreased swelling behaviour in comparison to methacylamide gels (gel-MOD) due to the presence of the more hydrophobic norbornene functionalities.

Furthermore, due to control of the number of reacted functionalities in thiol-ene hydrogels (gel-NB) by varying the thiol-ene ratio, unreacted norbornene functionalities can be obtained after crosslinking which can be applied for subsequent photografting of thiolated components (e.g. cell-interactive sequences, active pharmaceutical components, anti-oxidants, . . . ). However, by decreasing the thiol-ene ratio, the hydrogel material is characterized by even poorer mechanical properties, in combination with a higher water uptake capacity. As a consequence, the material can lose some of the benefits of high resolution additive manufacturing as post production swelling will increase the dimensions of the construct on the one hand, while also swelling induced stress inside the construct can lead to deformations. Furthermore, due to the poorer mechanical properties, the material might no longer be able to support its own weight when generating constructs with smaller feature sizes.

Another drawback of using thiol-ene hydrogels (gel-NB) is their limited storage stability at elevated temperatures during processing, which can be necessary for extrusion/deposition based additive manufacturing either with or without cell encapsulation, due to disulphide formation in the thiolated crosslinker. As a consequence, the material can either exhibit premature crosslinking, or the thiol-ene ratio is no longer controlled.

Jasper Van Hoorick et al. “Cross-Linkable Gelatins with Superior Mechanical Properties Through Carboxylic Acid Modification: Increasing the Two-Photon Polymerization Potential”, Biomacromolecules, vol. 18, no. 10, 29 Aug. 2017, pages 3260-3272 describes a particular bifunctional modified biopolymer referred to as GEL-MOD-AEMA comprising methacrylamide as first functional group and methacrylates as second functional group via reaction of the carboxylic acids with 2-aminoethylmethacrylate. This bifunctional modified biopolymer exhibits faster cross-linking kinetics compared to more conventional gel-MOD chain growth based biopolymers known in the art.

### SUMMARY OF THE INVENTION

It is an object of the present invention to provide modified biopolymer based polymers as for example modified gelatin avoiding the drawbacks of the prior art.

It is another object of the present invention to provide modified biopolymer based polymers that combine two functionalities: a first functionality enabling conventional free radical chain-growth polymerization and a second functionality susceptible to step-growth thiol-ene click reaction for example thiol-ene photoclick reaction.

It is a further object of the present invention to provide modified biopolymer based polymers that allow controlled post-crosslinking grafting after the free radical polymerization.

It is still a further object of the present invention to provide a modified gelatin combining the benefits towards material manipulation and (mechanical) stability of gelatin methacrylamide (gel-MOD) with the orthogonal click chemistry of gelatin-norbornene (gel-NB).

It is another object of the present invention to provide modified biopolymer based polymers suitable to prepare a hydrogel that allows local and controlled incorporation of certain functionalities by means of thiol-ene photografting.

It is another object of the present invention to provide a hydrogel having interesting mechanical properties such as strength and stiffness. In particular it is an object to provide a hydrogel having improved mechanical properties compared to gelatin-norbornene irrespective of the thiol-ene ratio while still exhibiting thiol-ene grafting potential.

It is still a further object of the present invention to provide a hydrogel with controllable swelling and/or water uptake capacity.

Additionally, it is an object of the present invention to provide a hydrogel with an improved storage stability in particular at elevated temperatures.

According to a first aspect of the present invention a bifunctional modified biopolymer based polymer is provided. The bifunctional gelatin comprises at least one polymer chain. The at least one polymer chain comprises at least two types of functional groups: n first functional groups and m second functional groups, with none of n or m being zero. The first functional groups comprise groups able of being radically cross-linked following a free radical chain-growth polymerisation. The second functional groups comprise thiol-ene cross-linkable groups that remain unreacted during free radical chain-growth polymerisation of said first functional groups.

Preferably, the bifunctional modified biopolymer based polymer has a degree of substitution for the first functional groups ranging between 1% and 95% and more preferably between 5% and 75%, for example between 15% and 75% or between 15% and 50%.

The bifunctional modified biopolymer based polymer, as for example bifunctional modified gelatin or bifunctional modified collagen, comprises at least one first functional group per polymer chain and preferably more than one first functional group per polymer chain. The bifunctional modified biopolymer based polymer, as for example the bifunctional modified gelatin, comprises at least one first functional group per polymer chain and comprises preferably more than one first functional group, for example 5, 10, 20, 30, 50, 60, 70, 80, 90 or 100 first functional groups per polymer chain. Preferably, the bifunctional modified biopolymer based polymer has a degree of substitution for the second functional groups ranging between 5% and 95% and more preferably between 5% and 75%, for example between 15% and 75% or between 15% and 50%.

The bifunctional modified biopolymer based polymer, as for example bifunctional modified gelatin or bifunctional modified collagen, comprises at least one second functional group per polymer chain and more preferably more than one second functional group per polymer chain. The bifunctional modified biopolymer based polymer, as for example the bifunctional modified gelatin, comprises at least one second functional group and comprises preferably more than one second functional group, for example 5, 10, 20, 30, 50, 60, 70, 80, 90 or 100 second functional groups per polymer chain.

The bifunctional modified biopolymer based polymer according to the present invention has the advantage to combine two functionalities: a first functionality enabling conventional free radical polymerization and a second functionality susceptible to thiol-ene click reaction, for example to thiol-ene photoclick reaction. The second functional groups remain unreacted during the free radical polymerization and allow to obtain post-crosslinking grafting. The second functional groups allow to introduce certain thiolated functionalities. The second functional groups allow for example post-processing grafting, such as post-processing grafting of bioactive molecules to further tailor the biopolymer based polymer towards specific needs.

The bifunctional biopolymer based polymer according to the present invention may comprise any type of biopolymer or polymeric biomolecule able to be functionalized with first and second functional groups. Biopolymers and polymeric biopolymers include polymers from a natural origin. For the purpose of this invention, the terms ‘biopolymer’ and ‘polymeric biomolecule’ are interchangeably used. For the purpose of this invention the term ‘biopolymer based polymers’ refers to all types of biopolymers, derivates of biopolymers, recombinant analogues of biopolymers, synthetic analogues of polymeric biopolymers.

Chemical derivates of biopolymers include but are not limited to biopolymers with a functionalized side chain as well as hydrolysis products of biopolymers.

Recombinant analogues of biopolymers include biopolymers which were obtained via encoding of a defined synthetic DNA sequence in an organism resulting in the synthesis of a biopolymer or protein with a defined amino acid sequence.

Synthetic analogues of biopolymers include polymers which were synthetically created by linking different monomers to each other resulting in a polymer containing different functionalities in its side chains. An example of such synthesis includes solid phase peptide synthesis.

Examples of biopolymer based polymers include polysaccharides, nucleic acids, gelatins, collagens, alginates, dextrans, agarose, glycosaminoglycans (for example hyaluronic acid), chitosans and carrageenans and derivates, recombinant analogues and synthetic analogues polysaccharides, nucleic acids, gelatins, collagens, alginates, dextrans, agarose, glycosaminoglycans (for example hyaluronic acid), chitosans and carrageenans.

For the purpose of the present invention biocompatible polymers are also considered as biopolymer based polymers. Particular preferred biopolymer based polymers comprise gelatin and collagen, recombinant gelatin and recombinant collagen.

The first functional groups may comprise any type of functional groups able or susceptible to radically cross-link following a free radical chain-growth polymerization. Preferred examples of first functional groups comprise methacrylamide functional groups, acrylamide functional groups, methacrylate functional groups and/or acrylate functional groups. Particularly preferred first functional groups comprise methaycrylamide functional groups and/or acrylamide functional groups. In particular embodiments the bifunctional modified biopolymer based polymer, as for example the bifunctional modified gelatin comprises only one type of first functional groups as for example methacrylamide functional groups or acrylamide functional groups or methacrylate functional groups or acrylate functional groups. In other embodiments the bifunctional modified biopolymer based polymer as for example the bifunctional modified gelatin comprises a combination of different first functional groups as for example a combination of methacrylamide functional groups and acrylamide functional groups.

The second functional groups may comprise any type of functional group that is able to or susceptible to thiol-ene cross-linking. Preferably, the second functional groups comprise functional groups able to or susceptible to thiol-ene crosslinking without being able to undergo competitive homopolymerisation. The second functional groups comprise for example norbornene functional groups, vinyl ether functional groups, vinylester functional groups, allyl ether functional groups, propenyl ether functional groups and/or alkene functional groups and/or N-vinylamide functional groups. Particularly preferred second functional groups comprise norbornene functional groups and/or vinylether functional groups. In particular embodiments the bifunctional modified biopolymer based polymer as for example the bifunctional modified gelatin comprises only one type of second functional groups as for examples norbornene functional groups or vinylether functional groups or vinyl ester functional groups or alkene functional groups or N-vinylamide functional groups. In other embodiments the bifunctional modified biopolymer based polymer as for example the bifunctional modified gelatin comprises a combination of different second functional groups as for example a combination of norbornene functional groups and vinylester functional groups.

In preferred embodiments the bifunctional modified biopolymer based polymer comprises methacrylamides as first functional groups and norbornene functional groups as second functional groups.

In other embodiments the bifunctional modified biopolymer based polymer comprises methacrylamides as first functional groups and vinylester functional groups as second functional groups.

In further embodiments the bifunctional modified biopolymer based polymer comprises acrylamides as first functional groups and norbornene functional groups as second functional groups.

In still further embodiments the bifunctional modified biopolymer based polymer comprises acrylamides as first functional groups and vinylester functional groups as second functional groups.

The bifunctional modified gelatin according to the present invention has preferably a total degree of substitution of the first functional groups and the second functional groups higher than 2%. With total degree of substitution of the first functional and the second functional groups is meant the sum of the degree of substitution of the first functional groups and the degree of substitution of the second functional group. The total degree of substitution ranges between 2% and 100% for example between 5% and 100% or between 5% and 95%, such as 20%, 40%, 50%, 60%, 70% or 80%.

The bifunctional modified biopolymer based polymer, as for example the bifunctional modified gelatin, comprises at least one first functional group per polymer chain and preferably more than one first functional group per polymer chain and comprises at least one second functional group per polymer chain and preferably more than one second functional group per polymer chain. The bifunctional modified biopolymer based polymer comprises for example 5, 10, 20, 30, 50, 60, 70, 80, 90 or 100 first functional groups and 5, 10, 20, 30, 50, 60, 70, 80, 90 or 100 second functional groups per polymer chain.

The bifunctional modified biopolymer based polymer according to the present invention may comprise one single polymer chain or may comprise a number of polymer chains. In any case a polymer chain comprises both first functional groups and second functional groups. By introducing the first functional groups and the second functional groups in one polymer chain, the biopolymer based polymer does not suffer from phase separation.

The bifunctional modified biopolymer based polymers according to the present invention are of particular importance to prepare hydrogels. The two functionalities of the biopolymer based polymers make them attractive for a high number of applications.

Bifunctional modified biopolymer based polymers allow for example the local and controlled incorporation of certain functionalities for example to obtain a better mimic for the natural extracellular matrix.

The bifunctional modified biopolymer based polymers also allow to introduce local and controlled zones of strength and/or stiffness by taking advantage of additional thiol-ene crosslinking.

Furthermore the bifunctional modified biopolymer based polymers allow straightforward material handling in combination with straightforward post production functionalization.

Additionally, the bifunctional modified biopolymer based polymers allow to control the final material water uptake capacity and solvent compatibility by post crosslinking grafting of hydrophilic or hydrophobic functionalities.

According to a second aspect of the present invention, a method to prepare a bifunctional modified biopolymer based polymer is provided. The method comprises the steps of
a) providing a biopolymer based polymer comprising at least one polymer chain, said polymer chain comprising primary functional groups;
b) functionalising a first part of said primary functional groups to introduce n first functional groups, with n not being zero, said first functional groups being able of being radically cross-linked following a free radical chain-growth polymerization;
c) functionalising a second part of said primary functional groups to introduce m second functional groups, with m not being zero, said second functional groups comprising thiol-ene crosslinkable groups.
wherein step b) and step c) can be performed simultaneously or wherein step b) can be performed before or after step c).

In preferred methods step b) is performed before step c). In alternative methods step c) is performed before step b). A method in which step c) is performed before step b) has the advantage to introduce a functionality prior to reaction with the first functional groups. This can be of importance to influence the hydrophobicity of the material prior to crosslinking or to introduce photoreversible groups via thiol-ene chemistry which can be cleaved after crosslinking to introduce zones of lower mechanical properties with spatiotemporal control.

The primary functional groups of the biopolymer based polymer comprise for example amine functional groups, for example primary amine functional groups, carboxylic acid functional groups, hydroxyl functional groups or a combination thereof.

In a preferred method the primary functional groups of the biopolymer based polymer comprise amine functional groups and step b) comprises a reaction of these amine functional groups or part of these amine functional groups for example with methacrylic anhydride.

In other preferred methods the primary functional groups of the biopolymer based polymer comprise carboxylic acid functional groups and step b) comprises a reaction of these carboxylic acid functional groups or part of these carboxylic acid functional groups.

In a further preferred method the primary functional groups of the biopolymer based polymer comprise hydroxyl functional groups and step b) comprises a reaction of these hydroxyl functional groups or part of these hydroxyl functional groups.

It is clear that in case the primary functional groups comprise a combination of primary functional groups, as for example a combination of amine functional groups, carboxylic acid functional groups and/or hydroxyl functional groups, step b) may comprise a combination of reactions, for example a reaction of the amine functional groups or part of the amine functional groups for example with methacrylic anhydride and/or a reaction of the carboxylic acid functional groups or part of the carboxylic acid functional groups and/or a reaction of the hydroxyl functional groups or part of the hydroxyl functional groups.

In another preferred method the primary functional groups of the biopolymer based polymer comprise amine functional groups and step c) comprises a reaction of these amine functional groups or of part of these amine functional groups for example with 5-norbornene-2-carboxylic acid. A preferred reaction of the amine functional groups or part of the amine functional groups uses carbodiimide coupling chemistry (for example using 1-ethyl-3-(3-dimethylamino)propyl)-carbodiimide hydrochloride (EDC)/N-hydroxysuccinimide (NHS)) to couple 5-norbornene-2-carboxylic acid to the amine functional groups.

In another preferred method the primary functional groups of the biopolymer based polymer comprise amine functional groups and step c) comprises a reaction of the amine functional groups or part of the amine functional groups with carbic anhydride.

In still a further preferred method the primary functional groups of the biopolymer based polymer comprise carboxylic acid functional groups and step c) comprises a reaction of these carboxylic acid functional groups or of part of these carboxylic acid functional groups for example with 5-norbornene-2-methylamine. A preferred reaction of the carboxylic acid functional groups or part of the carboxylic acid functional groups uses carbodiimide coupling chemistry to couple 5-norbornene-2-methylamine to the carboxylic acid functional groups.

In still a further preferred method the primary functional groups of the biopolymer based polymer comprise hydroxyl groups and step c) comprises a reaction of these hydroxyl functional groups or of part of these hydroxyl functional groups

It is clear that in case the primary functional groups comprise a combination of primary functional groups, as for example a combination of amine functional groups, carboxylic functional groups and/or hydroxyl functional groups, step c) may comprise a combination of reactions, for example a combination of the above described reactions, for example a combination of a reaction of the amine functional groups or of part of the amine functional groups for example with 5-norbornene-2-carboxylic acid for example using carbodiimide coupling chemistry and/or a reaction of the carboxylic acid functional groups or of part of these carboxylic acid functional groups for example with 5-norbornene-2-methylamine for example by using carbodiimide coupling chemistry and/or a reaction of the hydroxyl functional groups or of part of the hydroxyl functional groups.

In a particular preferred method the primary functional groups of the biopolymer based polymer comprise amine functional groups and step b) comprises a reaction of part of these amine functional groups for example with methacrylic anhydride whereas step c) comprises a reaction of part of these amine functional groups for example with 5-norbornene-2-carboxylic acid or step c) comprises a reaction of part of these amine functional groups with carbic anhydride.

In another particularly preferred method the primary functional groups of the biopolymer based polymer comprise carboxylic acid functional groups and step b) comprises a reaction of part of these carboxylic acid functional groups for example with 2-aminoethyl methacrylate whereas step c) comprises a reaction of part of these carboxylic acid functional groups for example with 5-norbornene-2-methylamine.

In further particularly preferred methods the primary functional groups of the biopolymer based polymer comprise amine functional groups and/or carboxylic acid functional groups and step b) comprises a reaction of part of these amine functional groups for example with methacrylic anhydride and/or a reaction of part of these carboxylic acid functional groups for example with 2-aminoethylmethacrylate whereas step c) comprises a reaction of part of the amine functional groups with for example 5-norbornene-2-carboxylic acid and a reaction of part of the carboxylic acid functional groups with for example 5-norbornene-2-methylamine.

A preferred method relates to a method of preparing a bifunctional modified gelatin. The method comprises the steps of
a) providing gelatin comprising at least one polymer chain, said polymer chain comprising primary functional groups as for example amine functional groups and/or carboxyl acid functional groups;
b) functionalising a first part of said primary functional groups, to introduce n first functional groups, with n not being zero, said first functional groups being able of being radically cross-linked following a free radical chain-growth polymerization;
c) functionalising a second part of said primary functional groups, to introduce m second functional groups, with m not being zero, said second functional groups comprising thiol-ene crosslinkable groups.
wherein step b) and step c) can be performed simultaneously or wherein step b) can be performed before or after step c). In preferred methods step b) is performed before step c). In alternative methods step c) is performed before step b).

The primary functional groups of gelatin comprise for example amine functional groups, for example primary amine functional groups, carboxylic acid functional groups, hydroxyl functional groups or a combination thereof.

In a preferred method the primary functional groups of gelatin comprise amine functional groups and step b) comprises a reaction of these amine functional groups or part of these amine functional groups for example with methacrylic anhydride.

In other preferred methods the primary functional groups of the gelatin comprise carboxylic acid functional groups and step b) comprises a reaction of these carboxylic acid functional groups or part of these carboxylic acid functional groups.

It is clear that in case the primary functional groups of gelatin comprise a combination of different functional groups, as for example amine functional groups and/or carboxylic acid functional groups and/or hydroxyl functional groups, step b) may comprise a combination of reactions, i.e. a reaction of the amine functional groups or part of the amine functional groups for example with methacrylic anhydride and/or a reaction of the carboxylic acid functional groups or part of the carboxylic acid functional groups for example with 2-aminoethyl methacrylate. If gelatin comprises further primary functional groups step b) may further comprise a reaction of these further primary functional groups or part of these further primary functional groups.

In another preferred method the primary functional groups of gelatin comprise amine functional groups and step c) comprises a reaction of these amine functional groups or of part of these amine functional groups for example with 5-norbornene-2-carboxylic acid. A preferred reaction of the amine functional groups or part of the amine functional groups uses carbodiimide coupling chemistry (for example using 1-ethyl-3-(3-dimethylamino)propyl)-carbodiimide hydrochloride (EDC)/N-hydroxysuccinimide (NHS)) to couple 5-norbornene-2-carboxylic acid to the amine functional groups.

In a further preferred method the primary functional groups of gelatin comprise carboxylic acid functional groups and step c) comprises a reaction of these carboxylic acid functional groups or of part of these carboxylic acid functional groups for example with 5-norbornene-2-methylamine. A preferred reaction of the carboxylic acid functional groups or part of the carboxylic acid functional groups uses carbodiimide coupling chemistry to couple 5-norbornene-2-methylamine to the carboxylic acid functional groups.

In another preferred method the primary functional groups of gelatin comprise amine functional groups and step c) comprises a reaction of the amine functional groups or part of the amine functional groups with carbic anhydride.

It is clear that in case the primary functional groups comprise a combination of amine functional groups and carboxylic acid functional groups, step c) may comprise a combination of reactions, i.e. a reaction or a combination of the above described reactions, for example a combination of a reaction of the amine functional groups or of part of the amine functional groups for example with 5-norbornene-2-carboxylic acid for example using carbodiimide coupling chemistry and a reaction of the carboxylic acid functional groups or of part of these carboxylic acid functional groups for example with 5-norbornene-2-methylamine for example by using carbodiimide coupling chemistry. If gelatin comprises further primary functional groups step c) may further comprise a reaction of these further primary functional groups or part of these further primary functional groups.

In a particular preferred method the primary functional groups of gelatin comprise amine functional groups and step b) comprises a reaction of part of these amine functional groups for example with methacrylic anhydride whereas step c) comprises a reaction of part of these amine functional groups with 5-norbornene-2-carboxylic acid or step c) comprises a reaction of part of these amine functional groups with carbic anhydride.

In another particularly preferred method the primary functional groups of gelatin comprise carboxylic acid functional groups and step b) comprises a reaction of part of these carboxylic acid functional groups whereas step c) comprises a reaction of part of these carboxylic acid functional groups with 5-norbornene-2-methylamine.

In further particularly preferred methods the primary functional groups of gelatin comprise amine functional groups and carboxylic acid functional groups and step b) comprises a reaction of part of these amine functional groups for example with methacrylic anhydride and a reaction of part of these carboxylic acid functional groups for example with 2-aminoethyl methacrylate whereas step c) comprises a reaction of part of the amine functional groups with 5-norbornene-2-carboxylic acid and a reaction of part of the carboxylic acid functional groups with 5-norbornene-2-methylamine.

A further preferred method relates to a method of preparing a bifunctional modified collagen. For the preparation of bifunctional modified collagen the same or similar methods as for the preparation of bifunctional modified gelatin can be considered.

According to a third aspect of the present invention a method to prepare a hydrogel is provided. The method comprises the steps of
a) providing bifunctional modified biopolymer based polymer, for example bifunctional modified gelatin or bifunctional modified collagen, as described above;
b) crosslinking said bifunctional modified biopolymer based polymer by free radical chain-growth polymerization of at least a part of said n first functional groups;
c) crosslinking and/or functionalizing at least a part of said m second functional groups.

An advantage of the method to prepare a hydrogel according to the present invention is that the bifunctional modified biopolymer based polymer can be crosslinked as specified in step b) while maintaining the crosslinking potential and/or the functionalizing potential as specified in step c).

Another advantage of the method to prepare a hydrogel according to the present invention is that crosslinking can be obtained in the absence of a thiolated crosslinker using free radical chain-growth polymerization in step b). Thiol-ene biopolymers or biopolymer based polymers as for example thiol-ene gelatin on the contrary require a thiolated crosslinker prior to crosslinking.

As the crosslinkable solution according to the present invention does not require a thiolated crosslinker, the crosslinkable solution remains more stable in comparison to thiol-ene crosslinkable biopolymers since some biopolymers (for example gelatin) needs to be heated above 30° C. or even above 40° C. to remain in solution. At temperatures above 30° C. disulphide formation can occur with the thiolated crosslinkers. This is considered as a considerable drawback of thiol-ene crosslinkable biopolymers as disulphide formation reduces the control over the number of reacted functionalities during crosslinking and results in even weaker hydrogels.

A further drawback of thiol-ene crosslinkable biopolymers or biopolymer based polymers is that the quantity of crosslinker needs to be calculated precisely to correspond to the number of ene functionalities which need to be crosslinked.

In a preferred method step b) comprises crosslinking in the presence of living cells including for example stem cells, cartilage cells, fibroblasts, . . . . To this purpose, a cell suspension inside a solution of the material prepared, followed by UV induced crosslinking, thereby not killing the suspended cells. As a result, a homogeneous cell distribution within the hydrogels can be obtained.

Step c) of the method to prepare a hydrogel may comprise either crosslinking or functionalizing or may comprise a combination of crosslinking and functionalizing by crosslinking a first part of the m functional groups and by functionalizing a second part of the m functional groups.

A particularly preferred type of functionalization comprises grafting, in particular photografting as for example using lithography and/or multiphoton assisted photografting (two-photon polymerization).

The hydrogel according to the present invention allows for example to introduce local zones of higher strength and/or zones of higher stiffness and this in a controlled way. This can for example be achieved by allowing a crosslinked hydrogel to swell inside a solution comprising a multifunctional thiol followed by localized grafting. The localized grafting can be performed using either a photomask or multiphoton lithography, thereby introducing zones of denser crosslinking.

Furthermore, the hydrogel allows local introduction of growth factors or cell adhesion zones (e.g. RGD sequences).

The functionalisation allows the introduction of active compounds, for example by covalent immobilization of an active compound using a thiol-ene mechanism. The active compounds comprise for example pharmaceutical compounds that may gradually be released upon degradation of the hydrogel.

Furthermore, by grafting hydrophilic groups (for example PEG) or hydrophobic groups (for example 7-mercapto-4-methylcoumarin) the water uptake capacity can be influenced.

According to a fourth aspect of the present invention a hydrogel, in particular a functionalized hydrogel, is provided.

According to a fifth aspect of the present invention the use of a hydrogel, in particular a functionalized hydrogel is provided.

A (functionalized) hydrogel according to the present invention is of particular importance in biomedical applications as for example tissue engineering. The (functionalized) hydrogel is for example adapted as wound dressing. The m second functional groups or part of the m second functional groups can furthermore provide an additional function.

As the crosslinkable solution obtainable from a bifunctional modified polymer according to the present has a high stability also at elevated temperature (above 30° C. or above 40° C.), the bifunctional modified polymer is suitable for 3D printing. This is an important advantage over hydrogels as for example thiol-ene hydrogels known in the art that as 3D printing of thiol-ene hydrogels is difficult because of their limited stability at elevated temperatures which may influence the material properties of the material.

### BRIEF DESCRIPTION OF THE DRAWINGS

The present invention will be discussed in more detail below, with reference to the attached drawings, in which:

FIG. 1 shows the storage modulus G′ (top) and the mass swelling ratio of different gelatin derivates (bottom) in equilibrium swollen state (all hydrogels were crosslinked at a 10 w/v % concentration in the presence of 2 mol % (relative to the amount of photocrosslinkable groups) Li-TPO-L photoinitiator;

FIG. 2 shows fluorescent microscopy images (left) and normal optical microscopy images (right) of the multiphoton assisted grafting of a fluorescent 7-methyl-4-mercaptocoumarin inside a crosslinked gel-MOD-NB pellet at different spatiotemporal energies;

FIG. 3 shows the cell viability using different gelatin concentrations for different gelatin derivates.

### DETAILED DESCRIPTION

The present invention will be described with respect to particular embodiments and with reference to certain drawings but the invention is not limited thereto but only by the claims.

### Example 1: Method to Prepare and Bifunctional Gelatin (Gel-MOD-NB)

### Materials

The following chemicals were used:
Gelatin type B, isolated from bovine hides by an alkaline treatment, provided by Rousselot (Ghent, Belgium).
Methacrylic anhydride, 5-norbornene-2-carboxylic acid, 1-ethyl-3-(3-dimethylamino)propyl)-carbodiimide hydrochloride (EDC), D,L-dithiotreitol (DTT) from Sigma-Aldrich (Diegem, Belgium).
Dimethyl sulfoxide (DMSO) (99.85%) and N-hydroxysuccinimide (98%) (NHS) purchased from Acros (Geel, Belgium).
Irgacure 2959 (1-[4-(2-hydroxyethoxy)-phenyl]-2-hydroxy-2-methyl-1-propane-1-one) from BASF.
Dialysis membranes Spectra/por (MWCO 12-14 kDa) were received from polylab (Antwerp, Belgium).

### Preparation of Gel-MOD

Gel-MOD with a DS (degree of substitution) of 72% was synthesized following a protocol described in A. I. Van Den Bulcke, B. Bogdanov, N. De Rooze, E. H. Schacht, M. Cornelissen, and H. Berghmans, “Structural and Rheological Properties of Methacrylamide Modified Gelatin Hydrogels,” Biomacromolecules, vol. 1, no. 1, pp. 31-38, March 2000 and according to the following reaction

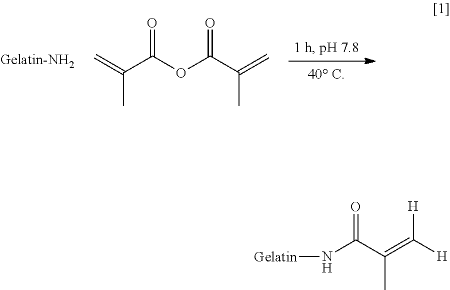

Briefly, 100 g of gelatin type B was dissolved in 1 L phosphate buffer (pH 7.8) at 40° C. After complete dissolution, 1 equivalent of methacrylic anhydride, relative to the primary amines present in the (hydroxy-)lysine and ornithine side chains, was added and the mixture was stirred vigorously. After 1 h, the mixture was diluted using 1 L of double distilled water (DDW) and introduced in dialysis membranes (Spectra/por MWCO 12-14 kDa) during 24 h against DDW. After dialysis, the pH of the mixture was adjusted to 7.4 to mimic natural ECM more closely using NaOH and gel-MOD was isolated using lyophilization (Christ freeze-dryer Alpha 2-4 LSC).

### Preparation of Gel-MOD-NB

For the preparation of 10 g gel-MOD-NB, first 5-norbornene-2-carboxylic acid was activated to its succinimidylester. To this end, first a 1.6 times excess of 5-norbornene-2-carboxylic acid (638 mg, 4.62 mmol), with respect to the EDC to be added was dissolved in 50 ml of dry DMSO (obtained via vacuum distillation using CaH₂ as drying agent). After complete dissolution, 0.75 equivalents of EDC (555 mg, 2.9 mmol) (relative to the original primary amines present in 10 g gelatin, i.e. 0.38 mmol/g gelatin) and 1.5 equivalents of NHS (relative to EDC) were added followed by 3 times degassing. The reaction was performed for at least 25 hours to eliminate any unreacted EDC functionalities which can result in gelatin crosslinking during the next reaction step.

After 25 h of reaction 10 g of gel-MOD with a known DS was dissolved in 150 ml dry DMSO (obtained via vacuum distillation using CaH₂ as drying agent) at 50° C. under inert atmosphere (Ar) and reflux conditions. After addition, the set-up was degassed 3 times and brought under Argon atmosphere. Following complete dissolution, the prepared 5-norbornene-2-succimidylester mixture was added to the gelatin solution followed by 3 times degassing. The mixture was allowed to react at 50° C. under inert atmosphere and reflux conditions for 5-20 h.

After the reaction, the mixture was precipitated in a tenfold excess of acetone, filtered on filter paper (VWR, pore size: 12-15 μm) using a Buchner filter, dissolved in DDW and dialysed (Spectra/por 4: MWCO 12-14 kDa) during 24 h at 40° C. against DDW. After dialysis, the pH was adjusted to 7.4 using NaOH followed by freezing and lyophilization (Christ freeze-dryer Alpha2-4 LSC). The preparation of gel-MOD-NB is illustrated by reaction [2]:

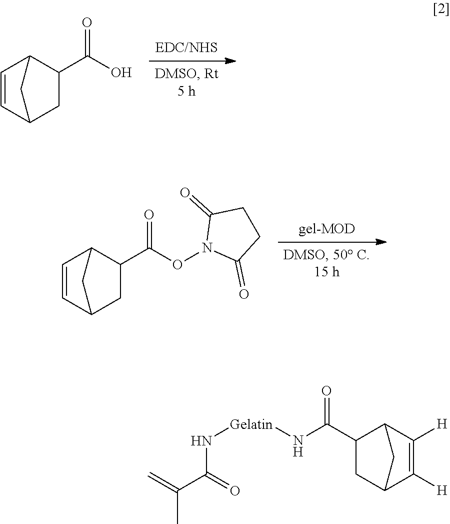

### Properties of Gel-MOD-NB

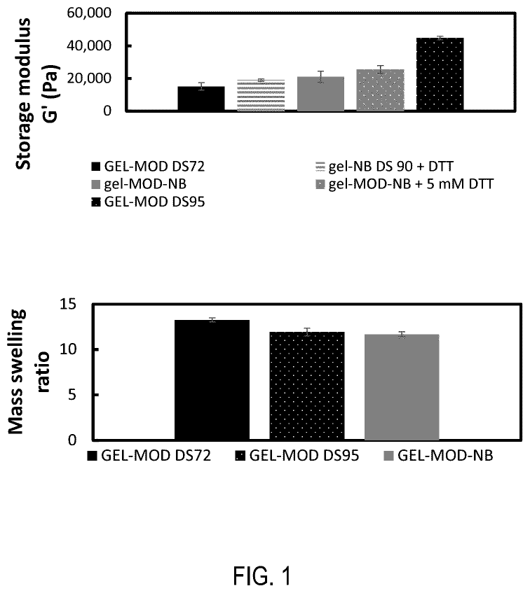

FIG. 1 shows the storage modulus G′ (top) and the mass swelling ratio of different gelatin derivates (bottom). The storage modulus G′ corresponds with the storage modulus of 10 w/v % crosslinked gelatin in equilibrium swollen state after 30 minutes of crosslinking (using 2 mol % (relative to the amount of crosslinkable functionalities) of Li-TPO-L as photoinitiator and 24 hours of incubation in milliQ for respectively gel-MOD DS 72, gel-NB DS 90+DTT (thiol/ene: 1), gel-MOD-NB DS 72 before and after an additional 30 minutes crosslinking in the presence of 5 mM DTT followed by equilibrium swelling and gel-MOD DS 95.

The mass swelling ratio of gel-MOD DS 72, gel-MOD DS 95 and gel-MOD-NB DS 72 is shown in the bottom panel of FIG. 1.

After crosslinking the first methacrymide functionalities and equilibrium swelling, the gel-MOD-NB derivative exhibits slightly higher stiffness in comparison to gel-MOD with a similar DS, although only the methacrylamides were polymerised. Although the inventors do not want to be bound by any theory, it is anticipated that this increase in mechanical properties is a consequence of the presence of hydrophobic norbornene functionalities which result in a lower water uptake capacity of the gel in comparison to the normal gel-MOD as can be derived from FIG. 1.

Furthermore, it should be noted that the gel-MOD-NB exhibits a higher stiffness in comparison to fully crosslinked gel-NB with a higher degree of substitution (e.g. 90%). Additionally, the mechanical properties of gel-MOD-NB are in between these of gel-MOD with a similar DS, but below the stiffness of gel-MOD which is fully functionalized (see FIG. 1). Furthermore, as proof of concept of the bifunctional nature, additional stiffness could be introduced after UV-irradiation in the presence of DTT after equilibrium swelling thereby benefitting from the thiol-ene photografting (see FIG. 1). However, still lower mechanical properties are obtained due to the nature of the formed additional crosslinks, since thiol-ene crosslinking results in a more homogeneous network, characterised by a lower crosslink density in comparison conventional chain-growth hydrogels.

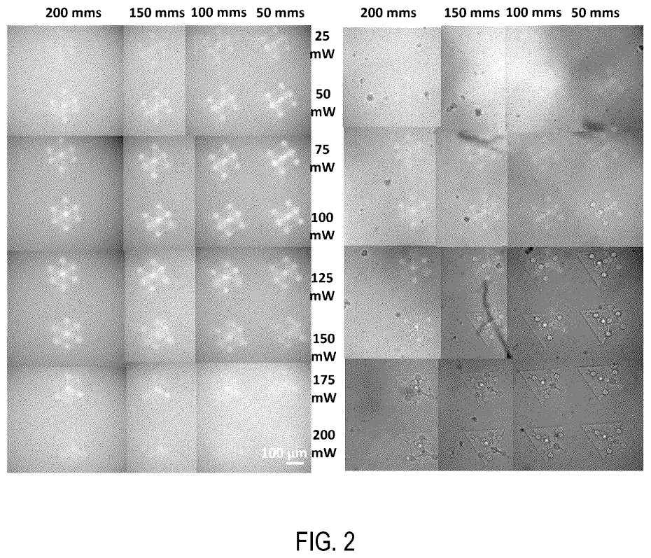

FIG. 2 shows the results of two-photon polymerization assisted photografting of a fluorescent 7-methyl-4-mercaptocoumarin inside a crosslinked bifunctional modified gelatin (gel-MOD-NB) pellet according to the present invention at different spatiotemporal energies, taking advantage of the norbornene functionalities.

The left picture of FIG. 2 shows fluorescent microscopy images. This images indicate the presence of coumarin with a high degree of spatiotemporal control.

The right picture of FIG. 2 shows normal microscopy images whereby the grafting of the coumarin leads to local shrinkage resulting in an observable difference in refractive index. It should be noted that besides no difference in refractive index is observed for all writing speeds at low laser power (e.g. 25 mW), the fluorescence microscopy clearly indicates successful grafting of the compound.

From FIG. 2 (left and right picture) can be derived that the bifunctional modified gelatin (gel-MOD-NB) allows post-production grafting with a high degree of spatiotemporal control thereby proving that the norbornene functionalities are not affected by the initial crosslinking step.

It should be noted that at high energies, grafting is less successful as a consequence of local overexposure thereby removing part of the material.

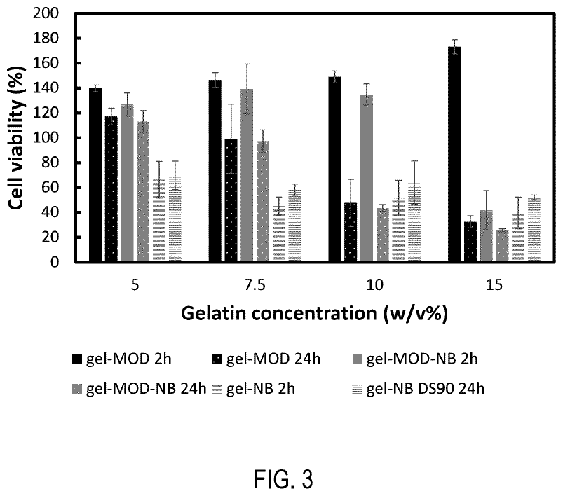

FIG. 3 shows the metabolic activity measured on confluent adipose tissue derived stem cells using a presto blue assay after 2 hours in the presence of different precursors and after 24 hours recovery in the absence of the different precursors. To this end, first a confluent monolayer of GFP labelled adipose tissue derived stem cells (passage 17) was obtained by seeding 100 μL of a 2 million cells/mL of medium per 96 well. Next, the cells were allowed to reach confluency after 24 hours of incubation. Next, 100 μL of a solution containing a hydrogel precursor was placed on top followed by another 2 hours of incubation. After 24 hours of incubation, the metabolic activity was measured using a presto blue assay, after which the material was removed from the well plate. Following another 24 hours of incubation, the metabolic activity was measured using a presto blue assay, as an indication of induced cell damage during the first 2 hours of incubation in the presence of a hydrogel precursor.

FIG. 3 indicates that bifunctional modified gelatin according to the present invention (gel-MOD-NB) exhibits a comparable cytotoxicity as gel-MOD, which can be considered as one of the gold standards in the field of tissue engineering and regenerative medicine. Additionally, in general higher cell viability is obtained in comparison to gel-NB, which is conventionally considered cytocompatible in literature.

### Example 2: Method to Prepare and Bifunctional Collagen (Col-MOD-NB)

### Preparation of Col-MOD

Col-MOD was synthesized by adapting a protocol described in A. I. Van Den Bulcke, B. Bogdanov, N. De Rooze, E. H. Schacht, M. Cornelissen, and H. Berghmans, “Structural and Rheological Properties of Methacrylamide Modified Gelatin Hydrogels,” Biomacromolecules, vol. 1, no. 1, pp. 31-38, March 2000 and according to the following reaction:

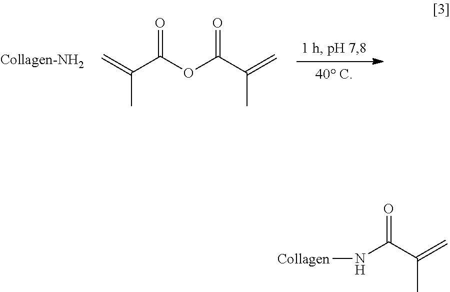

Briefly, 100 g of collagen was dissolved in 1 L phosphate buffer (pH 7.8) at 40° C. After complete dissolution, 1, 2 or 5 equivalent of methacrylic anhydride, relative to the primary amines present in the (hydroxy-)lysine side chains, was added and the mixture was stirred vigorously. After 1 h, the mixture was diluted using 1 L of double distilled water (DDW) and introduced in dialysis membranes (Spectra/por MWCO 12-14 kDa) during 24 h against DDW. After dialysis, the pH of the mixture was adjusted to 7.4 to mimic natural ECM more closely using NaOH and col-MOD was isolated using lyophilization (Christ freeze-dryer Alpha 2-4 LSC).

### Preparation of Col-MOD-NB

For the preparation of 10 g col-MOD-NB, first 5-norbornene-2-carboxylic acid was activated to its succinimydilester. To this end, first a 1.6 times excess of 5-norbornene-2-carboxylic acid, with respect to the EDC to be added was dissolved in 50 ml of dry DMSO (obtained via vacuum distillation using CaH₂ as drying agent). After complete dissolution, 0.75 equivalents of EDC (relative to the original primary amines present in 10 g collagen) and 1.5 equivalents of NHS (relative to EDC) were added followed by 3 times degassing. The reaction was performed for at least 25 hours to eliminate any unreacted EDC functionalities which can result in collagen crosslinking during the next reaction step.

After 25 h of reaction 10 g of col-MOD with a known DS was dissolved in 150 ml dry DMSO (obtained via vacuum distillation using CaH₂ as drying agent) at 50° C. under inert atmosphere (Ar) and reflux conditions. After addition, the set-up was degassed 3 times and brought under Argon atmosphere. Following complete dissolution, the prepared 5-norbornene-2-succimidylester mixture was added to the collagen solution followed by 3 times degassing. The mixture was allowed to react at 50° C. under inert atmosphere and reflux conditions for 5-20 h.

After the reaction, the mixture was precipitated in a tenfold excess of acetone, filtered on filter paper (VWR, pore size: 12-15 μm) using a Buchner filter, dissolved in DDW and dialysed (Spectra/por 4: MWCO 12-14 kDa) during 24 h at 40° C. against DDW. After dialysis, the pH was adjusted to 7.4 using NaOH followed by freezing and lyophilization (Christ freeze-dryer Alpha2-4 LSC). The preparation of col-MOD-NB is illustrated by reaction [4]:

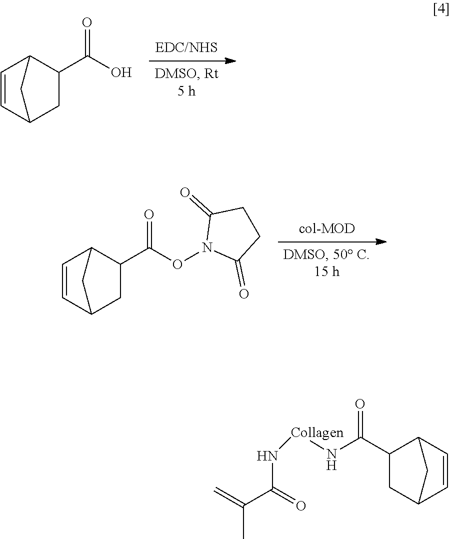

1. A bifunctional modified biopolymer based polymer, comprising at least one polymer chain, said at least one polymer chain comprising n first functional groups and m second functional groups, with n and m not being zero, said first functional groups comprising groups able of being radically cross-linked following a free radical chain-growth polymerisation and said second functional groups comprising groups able to thiol-ene cross-linking, said second functional groups remaining unreacted during free radical chain-growth polymerisation of said first functional groups.

2. A bifunctional modified biopolymer based polymer according to claim 1, wherein said biopolymer based polymer is selected from the group consisting of polypeptides, proteins, polysaccharides, nucleic acids, gelatins, collagens, alginates, dextrans, agarose, glycosaminoglycans, chitosans and carrageenans, derivates, recombinant analogues and synthetic analogues therefrom.

3. A bifunctional modified biopolymer based biopolymer according to claim 1 or claim 2, wherein said first functional groups comprise methacrylamide functional groups, acrylamide functional groups, methacrylate functional groups and/or acrylate functional groups.

4. A bifunctional modified biopolymer based biopolymer according to any one of the preceding claims, wherein said second functional groups comprise norbornene functional groups, vinylether functional groups, vinyl ester functional groups, allyl ether functional groups, propenyl ether functional groups, alkene functional groups and/or N-vinylamide functional groups.

5. A bifunctional modified biopolymer based polymer according to any one of the preceding claims, wherein said biopolymer based polymer comprises one polymer chain.

6. A method to prepare a bifunctional modified biopolymer based polymer as defined in any one of claims 1 to 5, said method comprising the steps of
(a) providing a biopolymer based biopolymer comprising at least one polymer chain comprising primary functional groups;
(b) functionalising a first part of said primary functional groups to introduce n first functional groups, with n not being zero, said first functional groups being able of being radically cross-linked following a free radical chain-growth polymerization;
(c) functionalising a second part of said primary functional groups to introduce m second functional groups, with m not being zero, said second functional groups comprising thiol-ene crosslinkable groups, said second functional group remaining unreacted during free chain-growth polymerization of said primary functional groups;
wherein step b) and step c) can be performed simultaneously or wherein step b) can be performed before or after step c).

7. A method according to claim 6, wherein said primary functional groups comprise amine functional groups and/or carboxylic acid functional groups and/or hydroxyl functional groups and step b) comprises a reaction of said amine functional groups and/or a reaction of said carboxylic acid functional groups and/or a reaction of said hydroxyl functional groups.

8. A method according to claim 6 or claim 7, wherein said primary functional groups comprise amine functional groups and/or carboxylic acid functional groups and/or hydroxyl functional groups and step c) comprises a reaction of said amine functional groups and/or a reaction of said carboxylic acid functional groups and/or a reaction of said hydroxyl functional groups.

9. A method according to claim 8, wherein said reaction of said amine functional groups and/or said reaction with said carboxylic acid functional groups and/or said reaction with said hydroxyl functional groups uses carbodiimide coupling chemistry.

10. A method to prepare a hydrogel, said method comprising the steps of
(a) providing a bifunctional modified biopolymer based polymer as defined in any one of claims 1 to 5,
(b) crosslinking said bifunctional modified biopolymer based biopolymer by free radical chain-growth polymerization of at least a part of said n first functional groups;
(c) crosslinking and/or functionalizing at least a part of said m second functional groups.

11. A method according to claim 10, wherein step c) comprises crosslinking of at least a part of said m second functional groups.

12. A method according to claim 10, wherein step c) comprises functionalizing at least part of said m functional groups.

13. A method according to claim 10, wherein step c) comprises crosslinking a first part of said m functional groups and functionalizing a second part of said m functional groups.

14. A hydrogel obtainable by a method as defined in any one of claims 10 to 13.

15. Use of a hydrogel as defined in claim 14 for tissue engineering and biofabrication.
